(19) United States
(12) Patent Application Publication
(10) Pub. No.: US 2005/0048659 A1
(43) Pub. Date: Mar. 3, 2005

(54) Method of preparing analytical sample, method of analyzing substance on surface of semiconductor substrate, and apparatus for preparing analytical sample

(75) Inventor: Yoshimi Shiramizu, Kanagawa (JP)

Correspondence Address: YOUNG & THOMPSON, 745 SOUTH 23RD STREET, 2ND FLOOR, ARLINGTON, VA, 22202, US

(73) Assignee: NEC ELECTRONICS CORPORATION, KANAGAWA (JP)

(21) Appl. No.: 10/923,794

(22) Filed: Aug. 24, 2004

(30) Foreign Application Priority Data
Aug. 28, 2003 (JP) 2003-304754

(51) Int. Cl. H01L 21/00; G01N 33/00

(52) U.S. Cl. 436/72

(57) ABSTRACT

An apparatus for preparing an analytical sample of a substance, which resides on the surface of a semiconductor substrate, capable of readily preparing an analytical sample of a substance which resides on the surface of a semiconductor substrate. A recovery solution is dropped on the surface of a silicon wafer. The recovery liquid can spread over the entire surface of the silicon wafer, because the surface property of the silicon wafer having an oxide film formed thereon is hydrophilic. The substance-to-be-analyzed which resides on the surface of the silicon wafer dissolves into the recovery liquid. Next, a hydrofluoric acid vapor is sprayed onto the surface of the silicon wafer. The oxide film is decomposed by the hydrofluoric acid vapor, and this turns the surface property of the silicon wafer into hydrophobic. Upon decomposition of the oxide film, the substance-to-be-analyzed dissolves into the recovery liquid. The hydrophobicity of the silicon wafer facilitates sampling of the recovery liquid.

This application is based on Japanese patent application No. 2003-304754, the content of which is incorporated hereinto by reference.

### BACKGROUND OF THE INVENTION

1. Field of the Invention

The present invention relates to a method of preparing an analytical sample, a method of analyzing substances on the surface of a semiconductor substrate, and an apparatus for analyzing substances on the surface of a semiconductor substrate.

2. Description of the Related Art

It has increasingly become important in the field of semiconductor manufacturing process to reduce metal contamination on the surface of semiconductor wafers with recent advancement in the degree of integration of semiconductor devices.

A clean process management is essential in order to prevent metal contamination on the surface of semiconductor wafers, and for this purpose, it is becoming more important to exactly obtain information on metal contaminants which reside on the surface of semiconductor wafers.

In a conventional procedure of exactly obtaining information on the metal contaminants which reside on the surface of semiconductor wafers, the metal contaminants are practically sampled and subjected to measurements.

A conventional method of measuring the metal contaminants which reside on the surface of semiconductor wafers will be briefed below.

The surface of the semiconductor wafer has a native oxide film or a thin oxide film formed thereon, and shows hydrophilicity.

In the measurement of the metal contaminants on the surface of the semiconductor wafer, first the native oxide or thin oxide film is decomposed using a hydrofluoric acid vapor or a concentrated hydrofluoric acid solution, to thereby make the surface of the semiconductor wafer hydrophobic.

Next, a recovery liquid (e.g., pure water, HF+H₂O₂, or HCl+H₂O₂) capable of maintaining the hydrophobicity of the surface of the semiconductor wafer is dropped on the, surface of the semiconductor wafer after being removed with the native oxide or thin oxide film to thereby have the hydrophobicity.

Next, the dropped recovery liquid is moved on the surface of the semiconductor wafer, to thereby allow any substances (metal contaminants, for example) which reside on the surface of the semiconductor wafer to be incorporated into the recovery liquid, and the recovery liquid having the substances which reside on the surface of the semiconductor wafer (analytical sample) is then recovered. It has been a general practice to subject thus-recovered recovery liquid to the analysis.

The reason why the surface property of the semiconductor wafer was initially converted into hydrophobic is to facilitate the recovery of the recovery liquid dropped on the semiconductor substrate.

The reason why the liquid capable of maintaining the hydrophobicity of the surface of the semiconductor wafer was used is that use of, for example, an oxidative liquid as the recovery liquid will undesirably form a native oxide on the surface of the semiconductor substrate and will convert the surface property of the semiconductor into hydrophilic, during the recovery process of metal contaminants and so forth by moving the oxidative recovery liquid over the surface of the semiconductor wafer, and this results in spreading of the recovery liquid over the entire surface of the semiconductor wafer and makes it difficult to recover the recovery liquid.

In an exemplary case where the liquid capable of maintaining hydrophobicity of the surface of the semiconductor wafer is used as the recovery liquid, and a silicon wafer is used as the semiconductor wafer, it was very difficult to decompose and recover elements (Cu and platinum group elements such as Pt and Ru) having ionization tendencies smaller than that of silicon. This is because the heavy metals having ionization tendencies smaller than that of silicon (Si) can readily be decomposed and recovered from the silicon wafer only when the oxidative liquid is used as the recovery liquid, since such heavy metals are adsorbed to the surface of Si, so that it is necessary to oxidize and dissolve the heavy metals in a drug solution.

Of various metal contaminants on the surface of the semiconductor wafer, only limited species of the metals were therefore measurable by the conventional technique using the liquid capable of maintaining the hydrophobicity of the surface of the semiconductor wafer as the recovery liquid.

Moreover, recent trends in use of a vast variety of metals as new materials in fabrication of semiconductor devices have raised another anticipation in that contamination of the semiconductor wafers can be caused also by these newly-used metals, not only by metals represented by Fe having already been in problem. This raises a new demand on a technique by which any metals being conventionally in problem and becoming newly used can be extracted at the same time in an efficient manner.

Japanese Laid-Open Patent Publication 1993-226443 discloses a method of extracting a contaminant, which is capable of extracting also elements having ionization tendencies smaller than that of silicon, by using a liquid incapable of maintaining hydrophobicity of the surface of semiconductor wafers.

More specifically, a hydrofluoric acid vapor is sprayed to the surface of a silicon wafer, aqua regia is dropped onto the surface of the silicon wafer having an oxide film already decomposed thereon by the hydrofluoric acid vapor, and the silicon wafer is heated, to thereby solubilize platinum, which is an element having an ionization tendency smaller than that of silicon, into aqua regia.

Heating of aqua regia on the silicon wafer inevitably makes the surface property of the silicon wafer hydrophilic due to formation of the native oxide film, so that the hydrofluoric acid vapor is again sprayed to the surface of the silicon wafer before aqua regia is recovered so as to make the surface property of the silicon wafer again hydrophobic, and aqua regia (analytical sample) is recovered.

Japanese Laid-Open Patent Publication 1998-332554 discloses a method of detecting contaminants on a silicon wafer, in which the surface of the wafer is exposed to a hydrofluoric acid vapor so as to allow the vapor to condense on the wafer surface, a recovery liquid is then dropped on the wafer so as to cover the entire surface thereof and to allow the droplet (hydrofluoric acid) dewed on the wafer surface to be recovered into the recovery liquid to thereby prepare a sample solution, and the sample solution (analytical sample) is collected.

Japanese Laid-Open Patent Publication 1995-280708 discloses a method of detecting contaminants on the surface of a silicon wafer using, as the recovery liquid, a liquid capable of converting the surface property of the semiconductor wafer into hydrophobic. More specifically, there is shown an example of using, as the recovery liquid, a surface treatment liquid containing predetermined concentrations of hydrofluoric acid and hydrogen peroxide.

The technique described in Japanese Laid-Open Patent Publication 1993-226443 involves spraying of the hydrofluoric acid vapor repeated twice, and this complicates the process of extracting the contaminants on the surface of the silicon wafer.

The method of extracting contaminants described in Japanese Laid-Open Patent Publication 1993-226443 may have surely facilitated the recovery of the recovery liquid because the recovery liquid is allowed to run on the surface of the silicon wafer after the surface property was converted into hydrophobic by spraying of the hydrofluoric acid vapor, but it has been difficult to spread the recovery liquid over the entire surface of the hydrophobic silicon wafer.

In contrast to this, the technique disclosed in Japanese Laid-Open Patent Publication 1998-332554 made it possible to spread the recovery liquid over the entire surface of the silicon wafer, because the surface of the wafer was exposed to a hydrofluoric acid vapor so as to allow the vapor to condense on the wafer surface, and a recovery liquid was then dropped on the wafer so as to cover the entire surface thereof.

The technique disclosed in Japanese Laid-Open Patent Publication 1998-332554, however, undesirably converts the surface property of the silicon wafer into hydrophobic before the recovery liquid is dropped onto the wafer surface if the hydrofluoric acid is supplied to the silicon wafer in an excessive amount, so that the recovery liquid is hardly spread over the entire surface of the silicon wafer. On the contrary, too small amount of the hydrofluoric acid vapor may undesirably leave a portion of the surface of the wafer unconverted into hydrophobic, and this makes it difficult to recover the recovery liquid. In other words, a high level of precision was required for controlling the amount of hydrofluoric acid to be supplied onto the wafer surface in the technique disclosed in Japanese Laid-Open Patent Publication 1998-332554.

The technique disclosed in Japanese Laid-Open Patent Publication 1995-280708, in which the surface treatment liquid containing predetermined concentrations of hydrofluoric acid and hydrogen peroxide is prepared, and thus-prepared surface treatment liquid is dropped on the surface of the silicon wafer, and makes it possible to more readily spread the surface treatment liquid over the entire surface of the silicon wafer.

The technique disclosed in Japanese Laid-Open Patent Publication 1995-280708 is, however, restricted in that the recovery liquid must be a liquid capable of converting the surface property of the semiconductor wafer into hydrophobic, and the restriction consequently limits a range of substances collectable from the surface of the semiconductor wafer.

### SUMMARY OF THE INVENTION

It is therefore objects of the present invention to provide a method of preparing an analytical sample of substances on the surface of a semiconductor substrate, capable of readily preparing an analytical sample of a wide variety of substances which reside on the surface of the semiconductor substrates; a method of analyzing substances which reside on the surface of a semiconductor substrate; and an apparatus for analyzing substances on the surface of a semiconductor substrate.

In consideration of accomplishing the aforementioned objects, a method of preparing an analytical sample of the present invention comprises a first step of supplying a recovery liquid for recovering a substance-to-be-analyzed to the surface of a semiconductor substrate having a hydrophilic film formed thereon; and a second step of supplying a vapor containing a substance capable of decomposing the hydrophilic film to the recovery liquid supplied to the surface of the semiconductor substrate.

Because the recovery liquid for recovering the substance-to-be-analyzed is supplied on the surface of the semiconductor substrate having the hydrophilic film formed thereon, the above-described invention makes it possible to readily spread the recovery liquid over the entire surface of the semiconductor substrate.

By supplying the vapor containing a substance capable of decomposing the hydrophilic film to the recovery liquid, the recovery liquid can recover contaminants and the substance-to-be-analyzed contained in the film during removal of the film with the aid of the vapor, and can recover also the substance-to-be-analyzed which resides on the surface of the semiconductor substrate having the film already removed therefrom. It is all enough to supply the vapor at least to an amount as much as being capable of making the entire surface property of the semiconductor substrate hydrophobic, so that it becomes less necessary to control the supply of the vapor so precisely.

Because the surface property of the semiconductor substrate becomes hydrophobic after the film is removed therefrom, the surface of the semiconductor substrate loses affinity to the recovery liquid, and this makes it possible to prevent the substance once recovered into the recovery liquid from re-adhering to the semiconductor substrate, and facilitates the recovery of the recovery liquid.

It is also made possible to use a liquid incapable of maintaining the hydrophobicity of the surface of the semiconductor wafer, in addition to the liquid capable of maintaining hydrophobicity of the surface of the semiconductor wafer and the liquid capable of converting the surface property of the semiconductor wafer into hydrophobic, and this facilitates the recovery of any substances which could not be recovered by using the liquid capable of maintaining hydrophobicity of the surface of the semiconductor wafer and the liquid capable of converting the surface property of the semiconductor wafer into hydrophobic. It is also made possible to extract metal contaminants, acidic components or ammonia component on the semiconductor substrate.

An apparatus for preparing an analytical sample of the present invention comprises a recovery liquid supply unit for supplying a recovery liquid for recovering a substance-to-be-analyzed to the surface of a semiconductor substrate having a hydrophilic film formed thereon; and a vapor supply unit for supplying a vapor containing a substance capable of decomposing the hydrophilic film to the recovery liquid supplied to the surface of the semiconductor substrate.

Because the recovery liquid for recovering the substance-to-be-analyzed is supplied on the surface of the semiconductor substrate having the hydrophilic film formed thereon by the recovery liquid supply unit, the invention makes it possible to readily spread the recovery liquid over the entire surface of the semiconductor substrate.

When the vapor supply unit supplies the vapor containing a substance capable of decomposing the hydrophilic film to the recovery liquid to thereby decompose the film, the recovery liquid recovers contaminants and the substance-to-be-analyzed contained in the film, and recovers also the substance-to-be-analyzed which resides on the surface of the semiconductor substrate having the film already removed therefrom. It is all enough to supply the vapor at least to an amount as much as being capable of making the entire surface property of the semiconductor substrate hydrophobic, so that it becomes less necessary to control the supply of the vapor so precisely.

Because the surface property of the semiconductor substrate becomes hydrophobic after the film is removed therefrom, the surface of the semiconductor substrate loses affinity to the recovery liquid, and this makes it possible to prevent the substance once recovered into the recovery liquid from re-adhering to the semiconductor substrate, and facilitates the recovery of the recovery liquid.

It is also made possible to use a liquid incapable of maintaining the hydrophobicity of the surface of the semiconductor wafer, in addition to the liquid capable of maintaining hydrophobicity of the surface of the semiconductor wafer and the liquid capable of converting the surface property of the semiconductor wafer into hydrophobic, and this facilitates the recovery of any substances which could not be recovered by using the liquid capable of maintaining hydrophobicity of the surface of the semiconductor wafer, or by the liquid capable of converting the surface property of the semiconductor wafer into hydrophobic. It is also made possible to extract metal contaminants, acidic components or ammonia component on the semiconductor substrate.

The hydrophilic film herein may be an oxide film, and the vapor containing a substance capable of decomposing the hydrophilic film may be a hydrofluoric acid vapor.

The recovery liquid may contain any one of HF+H₂O₂, HCl+H₂O₂, HNO₃+HCl, HClO₄, HNO₃, H₂O, H₂O₂, alkali aqueous solution and pure water.

The recovery liquid can more readily be recovered by spraying the hydrofluoric acid vapor from a plurality of positions so as to concentrate the recovery liquid in a single site.

It is also made possible to measure the substances which reside on the surface of the semiconductor substrate by, for example, a total reflection X-ray florescent analyzer, in the process of removing the solvent of the recovery liquid from the recovery liquid supplied with the vapor.

A method of analyzing a substance which resides on the surface of a semiconductor substrate of the present invention comprises analyzing the recovery solution (analytical sample) obtained by the above-described method of preparing an analytical sample.

Because the present invention utilizes an analytical sample obtained by the above-described method of preparing an analytical sample, the sample has been spread over the entire surface of the semiconductor substrate-to-be-analyzed, and to effectively recover the substance-to-be-analyzed, and this is successful in reducing labor of an operator in the analysis of substances which reside on the surface of the semiconductor substrate.

For the case where the recovery liquid is analyzed after being removed with the solvent, it is made possible to measure the substances on the semiconductor substrate by, for example, a total reflection X-ray fluorescent analyzer.

The present invention makes it possible to readily prepare an analytical sample of substances which reside on the surface of the semiconductor substrate. The reason will be described below.

Because the recovery liquid for recovering the substance-to-be-analyzed is supplied onto the surface of the semiconductor substrate having the hydrophilic film formed thereon, it is made possible to readily spread the recovery liquid over the entire surface of the semiconductor substrate.

Because the surface property of the semiconductor substrate becomes hydrophobic after the film is removed therefrom, the surface of the semiconductor substrate loses affinity to the recovery liquid, and this makes it possible to prevent the substance once recovered into the recovery liquid from re-adhering to the semiconductor substrate, and facilitates the recovery of the recovery liquid.

It is also made possible to use a liquid incapable of maintaining the hydrophobicity of the surface of the semiconductor wafer, in addition to the liquid capable of maintaining hydrophobicity of the surface of the semiconductor wafer and the liquid capable of converting the surface property of the semiconductor wafer into hydrophobic, and this makes it possible to recover any substances which could not be recovered by using the liquid capable of maintaining hydrophobicity of the surface of the semiconductor wafer, or by the liquid capable of converting the surface property of the semiconductor wafer into hydrophobic.

### BRIEF DESCRIPTION OF THE DRAWINGS

FIGS. 1A to 1F are explanatory drawings sequentially showing operational steps of a method of preparing an analytical sample according to an embodiment of the present invention.

### DETAILED DESCRIPTION OF THE INVENTION

The following paragraphs will describe embodiments of the present invention referring to the attached drawing.

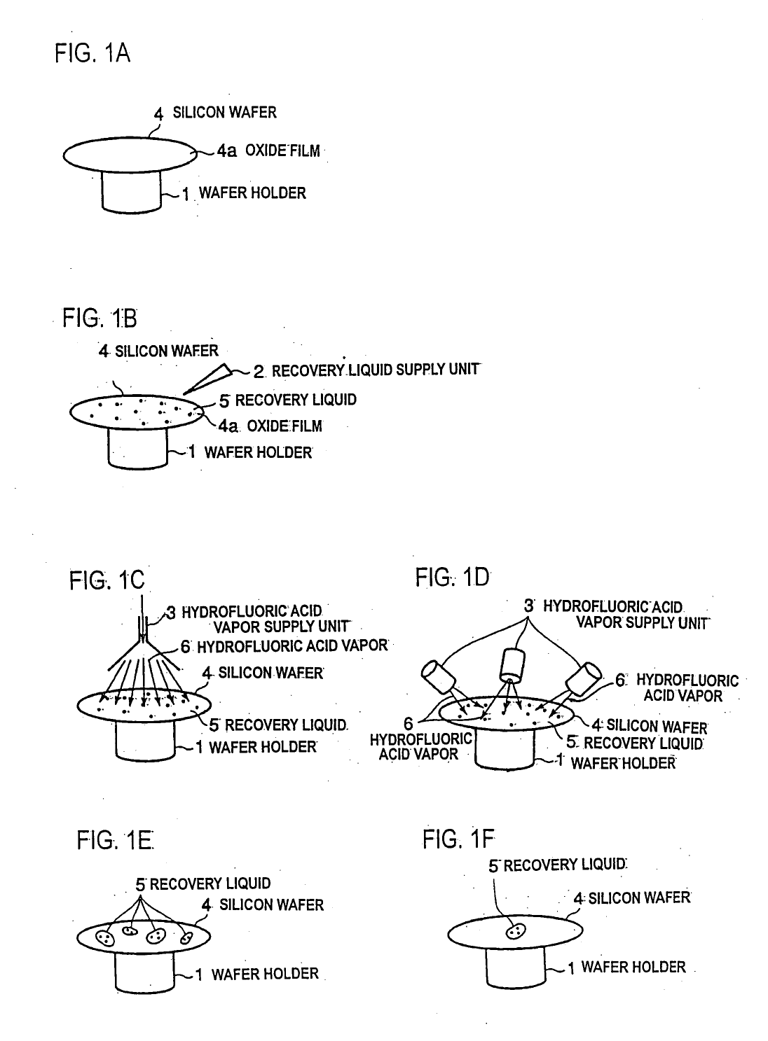

FIGS. 1A to 1F are an explanatory drawings of an apparatus for preparing an analytical sample according to an embodiment of the present invention, and a method of preparing an analytical sample according to an embodiment of the present invention.

The apparatus for preparing an analytical sample shown in FIGS. 1A to 1F includes a wafer holder 1, a recovery liquid supply unit 2, and a hydrofluoric acid vapor supply unit 3.

The wafer holder 1 holds a silicon wafer 4 as the semiconductor substrate. The silicon wafer 4 held by the wafer holder 1 (FIG. 1A) has an oxide film 4a such as native oxide or thin oxide film formed thereon. The surface of the silicon wafer 4 is hydrophilic because of hydrophilicity of the oxide film 4a formed on the surface of the silicon wafer 4.

The recovery liquid supply unit 2 supplies a recovery liquid 5 for recovering substances-to-be-analyzed onto the surface of the silicon wafer 4 having the oxide film 4a formed thereon. The hydrofluoric acid vapor supply unit 3 supplies a hydrofluoric acid vapor 6 to the recovery liquid 5 supplied onto the surface of the silicon wafer 4. The hydrofluoric acid vapor 6 can decompose the oxide film 4a.

Next paragraphs will explain a method of preparing an analytical sample of substances which reside on the surface of the semiconductor substrate according to a first embodiment, referring to FIGS. 1A to 1F.

The silicon wafer 4 having the oxide film 4a formed thereon is placed on the wafer holder 1 which is clean (FIG. 1A).

The clean recovery liquid 5 having a content of various metal contaminants suppressed to as low as 10 ppt or below and pure water having a content of various metal contaminants suppressed to as low as 1 ppt are prepared. The recovery liquid 5 is a liquid for recovering substances-to-be-analyzed which reside on the surface of the silicon wafer 4, and is appropriately selected depending on the species of the substances-to-be-analyzed.

In this embodiment, any one of HF+H₂O₂, HCl+H₂O₂, HNO₃+HCl, HClO₄, HNO₃ and H₂O is used as the recovery liquid 5. The recovery liquid 5 is used after being properly diluted with pure water.

The recovery liquid 5 in an amount of 1 ml to 5 ml is then dropped from the recovery liquid supply unit 2 onto the surface of the silicon wafer 4 so that the recovery liquid 5 can spread over the entire surface of the silicon wafer 4 showing hydrophilicity due to the oxide film 4a formed thereon (FIG. 1B). It is to be noted that the dropping of the recovery liquid 5 from the recovery liquid supply unit 2 may automatically be carried out, or may manually be carried out through operation of the recovery liquid supply unit 2 by an operator.

Because the surface of the silicon wafer 4 having the oxide film 4a formed thereon shows hydrophilicity, the recovery liquid 5 dropped on the surface of the silicon wafer 4 can readily spread over the entire surface of the silicon wafer 4 (more specifically, the surface-to-be-analyzed of the silicon wafer 4).

The substances-to-be-analyzed which reside on the surface of the silicon wafer 4 dissolve into the recovery liquid 5 spread over the entire surface of the silicon wafer 4.

Because the recovery liquid 5 is spread over the entire surface of the silicon wafer 4 in this embodiment, the substances-to-be-analyzed on the entire surface of the silicon wafer 4 can readily be recovered into the recovery liquid 5.

Next, the HF (hydrofluoric acid) vapor 6 is sprayed onto the surface of the silicon wafer 4, more specifically onto the recovery liquid 5 spread over the entire surface of the silicon wafer 4, from right above or from the direction oblique to the silicon wafer 4 (FIG. 1C or FIG. 1D).

FIG. 1C is an explanatory drawing showing an exemplary procedure of spraying the hydrofluoric acid vapor 6 from the hydrofluoric acid vapor supply unit 3 disposed right above the silicon wafer 4 onto the surface of the silicon wafer 4, more specifically onto the recovery liquid 5 spread over the entire surface of the silicon wafer 4.

FIG. 1D is an explanatory drawing showing an exemplary procedure of spraying the hydrofluoric acid vapor 6 from the hydrofluoric acid vapor supply units 3 disposed at a plurality of positions onto the surface of the silicon wafer 4, more specifically onto the recovery liquid 5 spread over the entire surface of the silicon wafer 4, so as to concentrate the recovery liquid 5 in a single site.

It is to be understood that a procedure of supplying the hydrofluoric acid vapor 6 to the recovery liquid 5 supplied onto the surface of the silicon wafer 4 is by no means limited to the exemplary case shown in FIG. 1C or FIG. 1D, and can properly be modified.

By spraying the hydrofluoric acid vapor 6 onto the surface of the silicon wafer 4, the oxide film 4a formed on the surface of the silicon wafer 4 is decomposed, and this makes the surface property of the silicon wafer 4 hydrophobic.

With the progress of decomposition of the oxide film 4a by spraying with the hydrofluoric acid vapor 6, the substances-to-be-analyzed which reside on the surface of the silicon wafer 4 dissolve into the recovery liquid 5.

Because the surface property of the silicon wafer 4 becomes hydrophobic after the oxide film 4a is removed therefrom, the surface of the silicon wafer 4 loses affinity to the recovery liquid 5, and this successfully prevents the substance once dissolved into the recovery liquid from re-adhering to the silicon wafer 4.

The surface property of the silicon wafer 4 becomes hydrophobic after being sprayed with the hydrofluoric acid vapor 6, and the recovery liquid(s) 5 then is (are) concentrated to a single site or several sites (see FIG. 1E or FIG. 1F).

FIG. 1E is an explanatory drawing showing an exemplary state of concentration of the recovery liquid 5, obtained after the hydrofluoric acid vapor 6 was sprayed onto the surface of the silicon wafer 4 from the hydrofluoric acid vapor supply unit 3 disposed right above the silicon wafer 4 as typically shown in FIG. 1C. In this case, the recovery liquid 5 is very likely to concentrate at several sites on the silicon wafer 4.

FIG. 1F is an explanatory drawing showing an exemplary state of concentration of the recovery liquid 5, obtained after the hydrofluoric acid vapor 6 was sprayed onto the surface of the silicon wafer 4 from a plurality of hydrofluoric acid vapor supply units 3 so as to concentrate the recovery liquid 5 at a single site on the silicon wafer 4, as typically shown in FIG. 1D. In this case, it is made possible to concentrate the recovery liquid 5 at a single site on the silicon wafer 4.

Next, the concentrated recovery liquid 5 is sampled, and the sampled recovery liquid 5 is analyzed by a trace metal analyzer such as those based on ICPMS (inductively coupled plasma mass spectrometry), atomic absorption and so forth. The recovery liquid 5 herein can readily be sampled by virtue of the hydrophobicity of the surface of the silicon wafer 4.

For the case where the recovery liquid 5 is concentrated at a single site on the surface of the silicon wafer 4, it is made possible to remove the solvent from the recovery liquid 5 by drying the concentrated recovery liquid 5, and the analytical sample comprising the recovery liquid 5 having its solvent already removed therefrom can be analyzed using a total reflection X-ray fluorescent analyzer.

According to this embodiment, it is made possible to use, as the recovery liquid, also a liquid incapable of maintaining the hydrophobicity of the surface of the semiconductor wafer, in addition to the liquid capable of maintaining hydrophobicity of the surface of the semiconductor wafer, and this widens a range of selectable recovery liquid, and also makes it possible to readily analyze metals which could not be analyzed or were very difficult to be analyzed. The metals which could not be analyzed or were very difficult to be analyzed include Au and Cu, and platinum group elements such as Ru, Pt, Ir. The reason why the metals which have been less likely to dissolve into the recovery liquid or very likely to adsorb to silicon become readily extractable from the silicon wafer is that decomposition of the oxide film by the hydrofluoric acid vapor proceeds in a system filled with the recovery liquid.

Another advantage resides in that efficiency of the analysis and efficiency of the preparation of the analytical sample can be improved to a considerable degree, because the elements which could not be analyzed or were very difficult to be analyzed can readily be extracted from the silicon wafer, together with Fe, Ni, Co, Zn, Al, Na and so forth, which have conventionally been extractable from the silicon wafer by spraying of hydrofluoric acid vapor.

Next, a second embodiment of the present invention will be explained.

The second embodiment of the present invention relates to an exemplary case where acidic components, ammonium components, alkali metals or alkali earth metals can be analyzed in a highly sensitive manner. Major differences of the second embodiment from the aforementioned example reside in components of the recovery liquid and method of analyzing the recovery liquid.

Next paragraphs will explain a method of preparing an analytical sample of substances which reside on the surface of the semiconductor substrate according to the second embodiment, referring to FIGS. 1A to 1F.

The silicon wafer 4 having the oxide film 4a formed thereon is placed on the wafer holder 1 which is clean (FIG. 1A).

Pure water, H₂O₂, an alkali aqueous solution such as aqueous sodium carbonate solution or the like is prepared as the recovery liquid 5. The recovery liquid 5 herein is a liquid for recovering the substances-to-be-analyzed which reside on the surface of the silicon wafer 4, and may be appropriately selected depending on whether the substances-to-be-analyzed is an acidic component, or an ammonium component, or an alkali metal, or an alkali earth metal, for example.

In an exemplary case where the substance-to-be-analyzed is an acidic component, a liquid available as the recovery liquid 5 may be any one of, or a mixture of pure water, pure water added with a trace amount of H₂O₂, and sodium carbonate solution.

In an exemplary case where the substance-to-be-analyzed is an ammonium component or an amine component, or an alkali metal or an alkali earth metal, a liquid available as the recovery liquid 5 may be either pure water or pure water added with a trace amount of H₂O₂.

The recovery liquid 5 in an amount of 1 ml to 5 ml is then dropped from the recovery liquid supply unit 2 onto the surface of the silicon wafer 4 so that the recovery liquid 5 can spread over the entire surface of the silicon wafer 4 showing a hydrophilicity due to the oxide film 4a formed thereon (FIG. 1B). It is to be noted that the dropping of the recovery liquid 5 from the recovery liquid supply unit 2 may automatically be carried out, or may manually be carried through operation of the recovery liquid supply unit 2 by an operator.

Because the surface of the silicon wafer 4 having the oxide film 4a formed thereon shows hydrophilicity, the recovery liquid 5 dropped on the surface of the silicon wafer 4 can readily spread over the entire surface of the silicon wafer 4 (more specifically, the surface-to-be-analyzed of the silicon wafer 4).

The substances-to-be-analyzed which reside on the surface of the silicon wafer 4 dissolve in the recovery liquid 5 spread over the entire surface of the silicon wafer 4.

Next, the HF (hydrofluoric acid) vapor 6 is sprayed onto the surface of the silicon wafer 4, more specifically onto the recovery liquid 5 spread over the entire surface of the silicon wafer 4, from right above or from the direction oblique to silicon wafer 4 (FIG. 1C or FIG. 1D).

By spraying the hydrofluoric acid vapor 6 onto the surface of the silicon wafer 4, the oxide film 4a formed on the surface of the silicon wafer 4 is decomposed, and this makes the surface property of the silicon wafer 4 hydrophobic.

With the progress of decomposition of the oxide film 4a by spraying with the hydrofluoric acid vapor 6, the substances-to-be-analyzed which reside on the surface of the silicon wafer 4 dissolve into the recovery liquid 5.

Because the surface property of the silicon wafer 4 becomes hydrophobic after the oxide film 4a is removed therefrom, the surface of the silicon wafer 4 loses affinity to the recovery liquid 5, and this successfully prevents the substance once dissolved into the recovery liquid from re-adhering to the silicon wafer 4.

The surface property of the silicon wafer 4 becomes hydrophobic after being sprayed with the hydrofluoric acid vapor 6, and the recovery liquid 5 then are concentrated to a single site or several sites (see FIG. 1E or FIG. 1F).

Next, the concentrated recovery liquid 5 is sampled, and the sampled recovery liquid 5 is subjected to an ion chromatography to analyze an acidic component as an anion, and ammonia and amine components as ammonium ion and alkali ion, respectively.

Analyzable components herein include hydrochloric acid ion, phosphoric acid ion, bromine ion, nitric acid/nitrous acid ion, sulfuric acid ion/sulfurous acid ion, Na, K, Ca, Li, Mg, ammonium ion, and various amines.

Where addition of H₂O₂ to the recovery liquid 5 herein is made the added H₂O₂ converts NOₓ, into nitric acid ion, and SOₓ into sulfuric acid ion, respectively, based on the oxidative action of H₂O₂ to facilitate to analyze those components.

Where use of an alkali aqueous solution such as aqueous sodium carbonate solution as the recovery liquid 5 herein is made the alkali aqueous solution raises solubility of the acidic component to facilitate to analyze such component.

The second embodiment makes it possible to analyze not only metal contaminant in the silicon wafer, but also molecular components contained in the silicon wafer.

It is to be understood that the present invention is by no means limited to the above-described embodiments, and allows various modifications.

1. A method of preparing an analytical sample comprising:
a first step of supplying a recovery liquid for recovering a substance-to-be-analyzed to the surface of a semiconductor substrate having a hydrophilic film formed thereon; and
a second step of supplying a vapor containing a substance capable of decomposing said hydrophilic film to said recovery liquid supplied to the surface of said semiconductor substrate.

2. The method of preparing an analytical sample according to claim 1, wherein
said hydrophilic film is an oxide film; and
said vapor containing a substance capable of decomposing said hydrophilic film is a hydrofluoric acid vapor.

3. The method of preparing analytical sample according to claim 2, wherein said recovery liquid contains any one of HF+H₂O₂, HCl+H₂O₂, HNO₃+HCl, HClO₄, HNO₃, H₂O, H₂O₂, alkali aqueous solution and pure water.

4. The method of preparing an analytical sample according to claim 1, wherein in said second step, said vapor containing a substance capable of decomposing said hydrophilic film is sprayed from a plurality of positions so as to concentrate said recovery liquid in a single site.

5. The method of preparing an analytical sample according to claim 4, further comprising removing the solvent from said recovery liquid supplied with said vapor containing the substance capable of decomposing said hydrophilic film.

6. The method of preparing an analytical sample according to claim 2, wherein in said second step, said vapor containing a substance capable of decomposing said hydrophilic film is sprayed from a plurality of positions so as to concentrate said recovery liquid in a single site.

7. The method of preparing an analytical sample according to claim 3, wherein in said second step, said vapor containing a substance capable of decomposing said hydrophilic film is sprayed from a plurality of positions so as to concentrate said recovery liquid in a single site.

8. A method of analyzing a substance which resides on the surface of a semiconductor substrate, comprising analyzing the analytical sample obtained by the method of preparing an analytical sample according to claim 1.

9. An apparatus for preparing an analytical sample comprising:
a recovery liquid supply unit for supplying a recovery liquid for recovering a substance-to-be-analyzed to the surface of a semiconductor substrate having a hydrophilic film formed thereon; and
a vapor supply unit for supplying a vapor containing a substance capable of decomposing said hydrophilic film to said recovery liquid supplied to the surface of said semiconductor substrate.

10. The apparatus for preparing an analytical sample according to claim 9, wherein
said hydrophilic film is an oxide film; and
said vapor containing a substance capable of decomposing said hydrophilic film is a hydrofluoric acid vapor.

11. The apparatus for preparing an analytical sample according to claim 10, wherein said recovery liquid contains any one of HF+H₂O₂, HCl+H₂O₂, HNO₃+HCl, HClO₄, HNO₃, H₂O, H₂O₂, alkali aqueous solution and pure water.

12. The apparatus for preparing an analytical sample according to claim 9, wherein said vapor supply unit is configured so as to spray said vapor containing a substance capable of decomposing said hydrophilic film from a plurality of positions.

13. The apparatus for preparing an analytical sample according to claim 10, wherein
said vapor supply unit is configured so as to spray said vapor containing a substance capable of decomposing said hydrophilic film from a plurality of positions.

14. The apparatus for preparing an analytical sample according to claim 11, wherein
said vapor supply unit is configured so as to spray said vapor containing a substance capable of decomposing said hydrophilic film from a plurality of positions.
